(19) United States
(12) Patent Application Publication
(10) Pub. No.: US 2014/0247331 A1
(43) Pub. Date: Sep. 4, 2014

(54) EYEWEAR TO ALLEVIATE EFFECTS OF MACULAR DEGENERATION

(71) Applicants: GUNTER A. HOFMANN, San Diego, CA (US); John L. Rogitz, San Diego, CA (US)

(72) Inventors: GUNTER A. HOFMANN, San Diego, CA (US); John L. Rogitz, San Diego, CA (US)

(21) Appl. No.: 13/782,332

(22) Filed: Mar. 1, 2013

(51) Int. Cl. A61F 9/08; G02C 7/02

(52) U.S. Cl. CPC A61F 9/08; G02C 7/022
USPC 348/62; 351/159.01; 351/159.58; 351/57

(57) ABSTRACT

A system includes human-wearable eyewear that utilizes an imager in communication with displays via a microprocessor to transform the central pixels of an image into a ring shaped image that may be presented on the displays. Patients with macular degeneration may be enabled to visualize the central pixels of an image using their peripheral vision. Various lenses are also disclosed for providing an optical-only solution for producing a ring-shaped image.

### FIELD OF THE INVENTION

The present application relates generally to human-wearable eyeware to alleviate the effects of macular degeneration.

### BACKGROUND OF THE INVENTION

A patient suffering from macular degeneration loses his central vision before losing his peripheral vision, effectively blinding the patient. The symptoms of macular degeneration are sought to be cured, but to date no absolute cure exists and damage done by the disease cannot be reversed.

### SUMMARY OF THE INVENTION

Present principles recognize there may be alternatives to curing the disease such as focusing images onto the functional, peripheral portions of the eye, thereby allowing macular degeneration patients to perceive objects in front of them.

An apparatus configured to redirect light onto a patient's peripheral vision eye location includes human-wearable eyeware frame that supports an input element onto which a light beam impinges, a transition member receiving light from the input element, and an output element. The input surface and transition member cooperate to spread the light into a ring-shaped pattern. The output element then receives the ring-shaped pattern and presents a human-visible representation thereof.

The apparatus may be embodied as human-wearable eyeglasses. The light beam can define a first radius and the ring-shaped pattern can define a second radius larger than the first radius. The ring-shaped pattern may be a substantially hollow ring such that substantially all of the light beam can be spread into the substantially hollow ring. The input element may transform light into electrical signals and the transition member can include a processor programmed to spread a digital representation of the electrical signals from a solid circular pattern to a hollow ring shaped-pattern.

The input element may be a first surface of a lens and the output element can be a second surface of a lens. The transition member can be defined by one or more optical components arranged between the surfaces. The first surface may be concave, may include plural prisms, and/or may be established at least in part by a cuspate surface. The second surface can be convex. The first surface and second surface may be defined by a common lens or can be defined by respective lenses.

In another aspect, an electro-optical apparatus is wearable by a person to direct incoming light in a substantially solid pattern into a hollow ring perceivable by peripheral vision of the person. The apparatus has a processor and at least one imager receiving the incoming light and sending signals representative thereof to the processor. One or more output elements such as matrix displays controlled by the processor visibly present representations of at least some of the signals in the hollow ring.

In another aspect, a lens includes a substrate and concentric rings of Fresnel ridges formed on the substrate. The spacing between adjacent concentric Fresnel ridges becomes progressively less from the perimeter of the lens toward the center of the lens. Also, slopes relative to an axis of light entering the lens of non-vertical sides of the ridges become progressively steeper, ridge to ridge, from the perimeter of the lens to the center of the lens, such that light entering the lens is diverted into a hollow ring-shaped pattern of light exiting the lens.

In another aspect, concentric rings of Fresnel ridges are formed on a thin flexible substrate configured for being held onto an outer surface of an eyeglass lens by adhesive or by simple friction/static charge. The Fresnel ridges have a configuration such that light impinging at and near the center of the lens is redirected radially outwardly into a hollow ring, whereas light impinging on outer portions of the lens is allowed to propagate into the hollow ring without substantial redirection. The configuration of the Fresnel ridges may focus substantially most or all of the light incident on the lens into the hollow outer ring. In this way, the configuration of the Fresnel ridges is established such that the width of the hollow ring substantially matches a remaining width of peripheral vision of a patient suffering from macular degeneration.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a schematic diagram of a non-limiting example of human-wearable eyeglass frames in accordance with present principles;

FIG. 2 is a perspective view of a cuspate lens;

FIG. 3 is a schematic view, partly in cross-section, of the lens of FIG. 2 receiving light from a parallel light source across line 2-2;

FIG. 4 is a cross section of a lens in which the concentrating prisms run in parallel straight lines and the supplementary spreading prisms are in parallel straight lines perpendicular to the first prisms and form a square or diamond shaped design;

FIG. 5 is a block diagram of an electro-optical embodiment;

FIG. 6 is a flow chart of example logic;

FIG. 7 is a schematic diagram showing mapping incoming light into an outer hollow ring;

FIG. 8 is plan view of an alternate optical-only embodiment, showing a thin substrate with a Fresnel lens pattern on it to spread light into a hollow ring;

FIG. 9 is a cross-section taken along the line 9-9 in FIG. 8, i.e., FIG. 9 shows one half of the diameter of the lens in cross-section elevation view, showing that the substrate may be placed over the outer surface of a conventional glass lens;

FIG. 10 is another elevation view of a part of the lens shown in FIG. 8, juxtaposed with a portion of the cuspate lens shown in FIGS. 2 and 3 to illustrate the relationship between groove spacing and configuration in the Fresnel version versus slope of the cuspate lens; and

FIG. 11 is a plan view of the ring into which light is focused by the lens of FIGS. 8-10.

### DETAILED DESCRIPTION OF THE PREFERRED EMBODIMENT

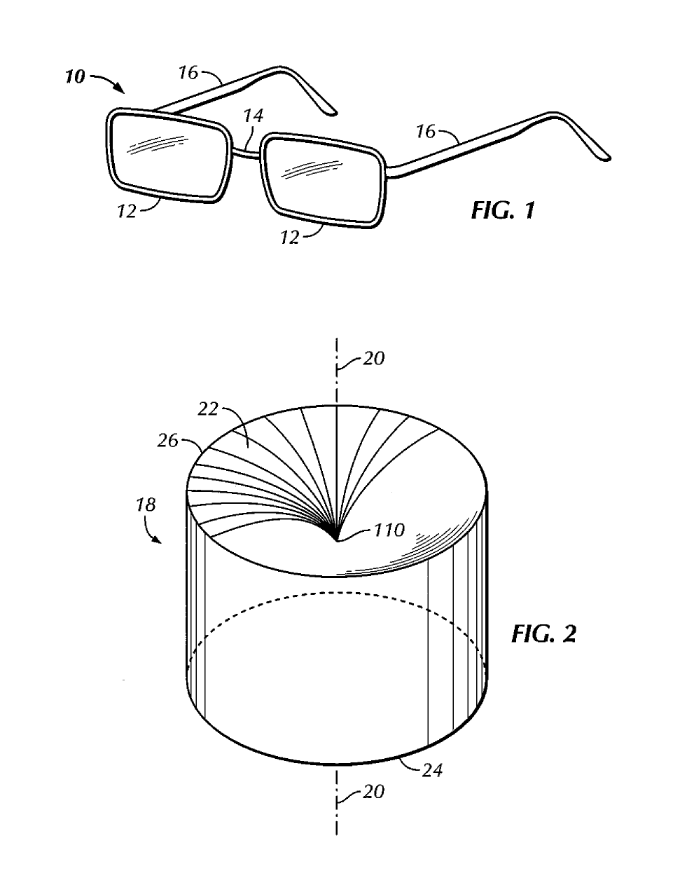

Referring initially to FIG. 1, a schematic diagram of a human-wearable eyeglass frame, generally designated 10, is shown and includes left and fight focusing assemblies 12. The frame 10 material may be made of durable material such as, but not limited to, fiberglass, nylon, zyl, or other plastic. The focusing assemblies 12 are medially connected by a connector piece 14 that may or may not be composed of the same material as the rest of the eyeglass frame 10. Left and right foldable arms 16 may be included as part of the frame 10 and can be connected by hinges to the lateral aspects of the focusing assemblies 12.

The focusing assemblies 12 may be established by optical components exclusively or by electro-optical assemblies. FIGS. 2 and 3 illustrate a focusing assembly 12 embodied by an example of the former. A refractive device or lens 18 is shown and can be made by lathing or molding of an optically transmissive material such as, for example, glass, polymethylmethacrylate or the like. The lens 18 has a central longitudinal axis 20, front surface 22, and rear surface 24. The front surface 22 extends laterally from the axis 20 toward a circular periphery 26 and is radially symmetric with respect to the axis 20. The front surface 22 is formed with a cusp 28 on the axis 20. The slope magnitude is greatest at the cusp 28 and decreases from the cusp 28 to a minimum at the periphery 26.

As best shown in FIG. 3, the lens 18 receives incident light I₀ oriented parallel with respect to the axis 20. The light is bent or refracted at the front surface 22 as shown. It is to be understood that the path of the light through the lens 18 depends on the angle of the surface 22 through which the light is received and the refractive index of the lens material. The decreasing slope magnitude of the surface 22 generally refracts the light away from the axis 20. For example, path A followed by the light received at the surface 22 adjacent to the cusp 28 is bent more sharply away from the axis 20 than the light received at the surface 22 adjacent to the periphery 26 which follows a path B substantially parallel to the axis 20. The result is that the light E transmitted through the surface 24 is generally refracted away from the axis 20 producing a darkened central circle 30 from which the light E is generally excluded and a generally bright ring 32 into which the light is directed. The lens 18 may or may not have a focal plane, depending on the geometry of the front surface 22, the refractive properties of the material of which the lens 18 is made, and the geometry of the rear surface 24. Additional details of the embodiment shown in FIGS. 2 and 3 are set forth in U.S. Pat. No. 4,834,484, incorporated herein by reference.

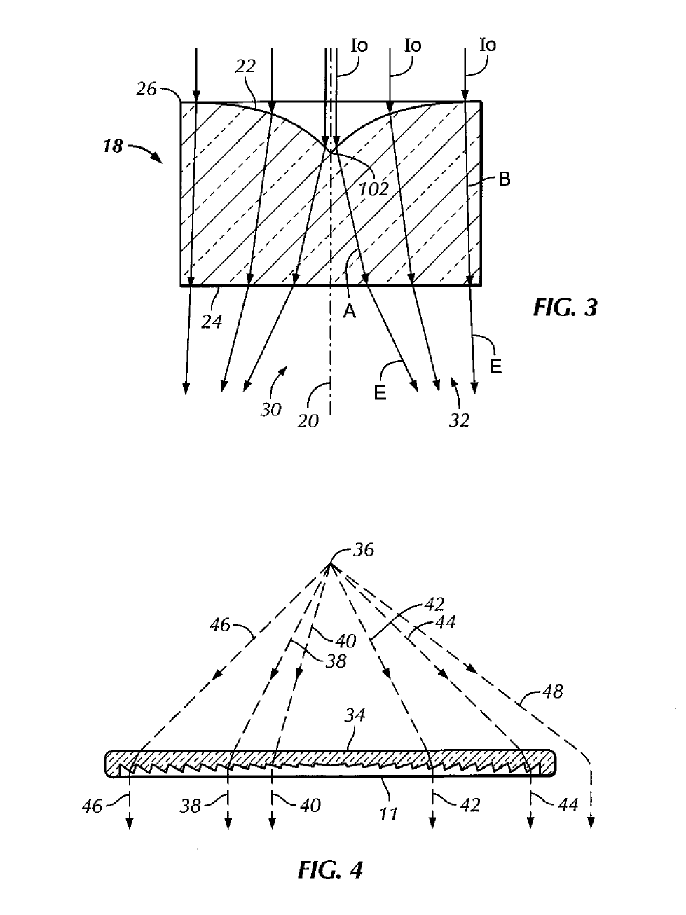

FIG. 4 illustrates another optical-only embodiment of a focusing assembly 12, incorporating the light spreading prisms. A light source is shown at 36. This section includes some of the prisms 34 on the inside surface, or input surface, of a convex lens. These act to reduce the angle of incidence at the inner surface and thereby decrease the deviation obtained at the inner surface and to increase the deviation obtained at the outer surface, or output surface, so that the net result is a spreading of the light rays which come through this portion of the lens in the plane shown in FIG. 4. Typical light rays 38, 40, 42, and 44 indicate this spreading effect. Light rays 46 and 48 outside of the area covered by the interior prisms are not changed in direction and such light has only the normal spread of the light from the light source itself. Thus the use of the inside prisms 34 produces a greater spread of light in directions parallel to the prisms on the outer surface than would otherwise be obtained. The resulting light distribution is concentrated completely in one set of parallel planes and is spread to a wide degree in the parallel planes at right angles. A lens with a concave outer surface may receive light exiting the prisms on an inner surface opposite the concave outer surface to focus the light into a ring-shaped pattern. Additional details of the embodiment shown in FIG. 4 are set forth in U.S. Pat. No. 2,082,100, incorporated herein by reference.

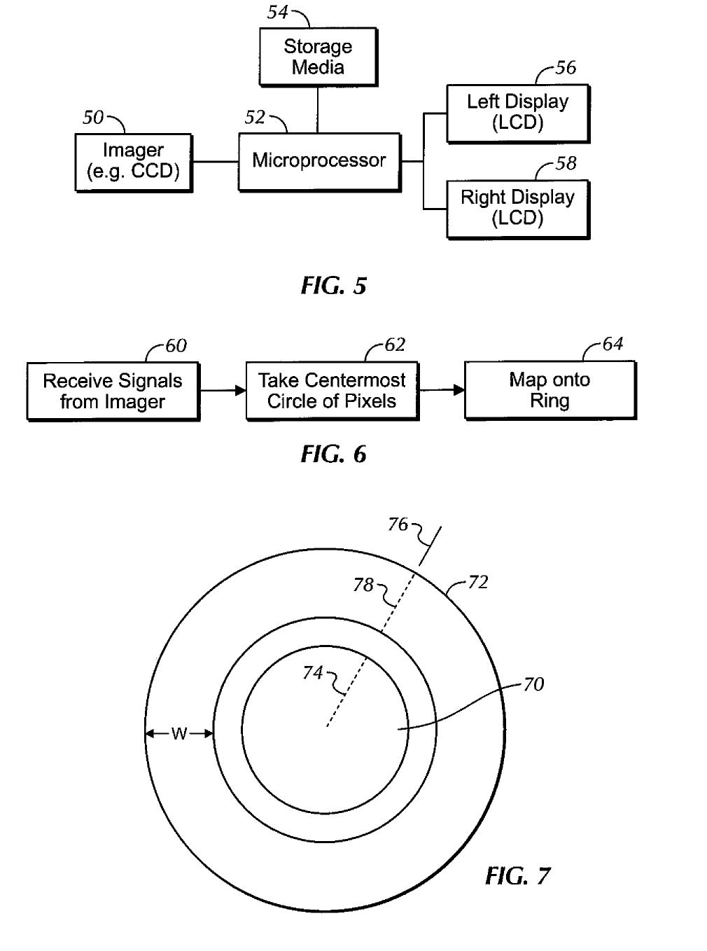

FIGS. 5 and 6 illustrate an electro-optical embodiment of the focusing assemblies 12 shown in FIG. 1. An imager 50, such as, but not limited to, a CCD imager, establishes an input surface and receives incoming photons and converts them into electron charges that are processed by appropriate circuitry and communicated to a microprocessor 52. The microprocessor 52 establishes a transition member and may access instructions stored on a computer readable storage medium 54 such as disk-based or solid state storage to execute logic herein. The microprocessor 52 outputs image signals to a left display 56 and a right display 58. The left and right displays 56, 58 establish an output surface and may be matrix-type displays such as liquid crystal diode (LCD) displays or light emitting diode (LED) displays mounted into left and right lens rims of the eyeglass frame 10 in FIG. 1 to establish portions of the focusing assemblies 12, respectively, of the eyeglass frame 10. Note that the imager 50 may be mounted on the element 14 in FIG. 1 to receive incoming light and the microprocessor with storage medium may be supported at any convenient location on the frame of the eyeglasses.

FIG. 6 diagrams example logic for the execution of instructions stored on the storage media 54 by the microprocessor 52 and begins with the microprocessor 52 receiving signals from the imager 50 at block 60. The microprocessor 52 may distinguish the centermost circle of pixels at block 62 and map them onto the displays 56, 58 in the shape of respective hollow rings at block 64. Patients with macular degeneration experience difficulty focusing the center, circular-shaped portion of a perceived image. Thus, mapping the centermost pixels received by the imager 50 in the form of a ring onto displays 56, 58 effectively allows patients with macular degeneration to see the center, circular-shaped portions of images in the form of a ring using their peripheral vision.

FIG. 7 illustrates the above logic and divulges additional processing details that may be employed. Light is received from in front of the wearer of the glasses typically spread to fill a center circle pattern 70. The light is converted into pixels as described above and mapped into a hollow ring-shaped pattern 72 for display on the LCDs 56, 58. The width “w” of the ring-shaped pattern 72 may be established by programming of the processor to approximate the width of a particular patient's remaining peripheral vision. Thus, patients with greater peripheral vision can be fitted with glasses in which the ring-shaped pattern 72 has a relatively wide width, whereas patients with less peripheral vision can be fitted with glasses in which the ring-shaped pattern 72 has a relatively small width, to better match the glasses with the patient.

Pixels derived from the center circle pattern 70 must be mapped into the ring-shaped pattern 72. In one example, pixels along a radial in the center circle pattern 70 such as pixels 74 along a radial 76 are mapped to pixel locations 78 in the ring-shaped pattern 72, arranged along the same radial 76. When the width “w” of the ring-shaped pattern 72 is equal to the radius of the center circle pattern 70, the mapping may be one-to-one, i.e., if N pixels lie along the radial 76 within the center circle pattern 70, these N pixels will be mapped to N corresponding pixel positions in the ring-shaped pattern 72 along the radial 76. On the other hand, when the width “w” of the ring-shaped pattern 72 is less than the radius of the circle 70, not all N pixels along the radial 76 within the circle 70 will be mapped to the ring-shaped pattern 72 along the radial 76. To select which of the N pixel(s) in the circle 70 will not appear in the ring-shaped pattern 72, every other pixel may be omitted when the width “w” of the ring-shaped pattern 72 is one-half the radius of the circle 70, or every third pixel may be omitted when the width “w” of the ring-shaped pattern 72 is two-thirds of the circle 70, and so on. Or, the pixel values along one or more radials may be averaged, and pixels with values with the greatest deviation from the average value may be omitted from the ring-shaped pattern 72, from greatest deviation first, to next greatest deviation, and so on until only sufficient pixels remain to completely fill the width of the ring-shaped pattern 72.

Yet again, the opposite heuristic may be used. That is, the pixel values along one or more radials may be averaged, and pixels with values with the least deviation from the average value may be omitted from the ring-shaped pattern 72, from least deviation first, to next least deviation, and so on until only sufficient pixels remain to completely fill the width of the ring-shaped pattern 72.

In the case in which the width “w” of the ring-shaped pattern 72 is greater than the radius of the circle 72, additional pixels may be generated based on those along a radial in the circle 70 to completely fill the pixel positions along the corresponding radial in the ring-shaped pattern 72. This may be done by interpolation, e.g., when only N pixels are arranged along a radial in the circle 70 but owing to w wide width “w” in the ring-shaped pattern 72, N+M pixel locations are available to be filled in the ring-shaped pattern 72, either some pixel locations in the ring-shaped pattern 72 may be left unfilled or additional pixel values may be generated by interpolating a value between first and second adjacent pixel values and then inserting a pixel with the interpolated value between the first and second pixel values in the ring-shaped pattern 72.

The same principles may be used between adjacent radials. Since the distance between radials spread from the circle 70 to the ring-shaped pattern 72, the pixel values along a first radial in the circle 70 can be averaged, on a pixel-by-pixel basis, with pixel values along a second, immediately adjacent radial in the circle 70, with the pixels being averaged with other pixels of the same distance from the center of the circle 70. The resulting new line of pixels may then be inserted between the radials in the ring-shaped pattern 72 corresponding to the first and second radials in the circle 70. In this way, the effect of geometric spreading between the circle 70 and ring-shaped pattern 72 is accounted for.

FIGS. 8-10 show an alternate lens 100 which may be implemented by forming concentric and in some embodiments circular rings of Fresnel ridges 102 on a thin flat substrate such as a flexible plastic substrate that may be held onto the outer surface of a conventional eyeglass lens 104 by adhesive or by simple friction/static charge. The periphery of the lens 100 may be round as shown, so that the lens is a flat disc. The periphery may assume other shapes generally to confirm to an eyeglass lens on which the disc may be placed for adherence by friction or adhesive. Thus, the periphery of the lens 100 may be ovular or rectilinear or other shape.

Referring briefly to FIG. 11, the lens 100 focuses light impinging at and near the center of the lens radially outwardly into a hollow ring “R” the width “W” of which is established by the configuration of the ridges described below to match the remaining width of the peripheral vision of a patient suffering from macular degeneration. Light impinging on the outer portions of the lens 100 (FIGS. 8-10) is allowed to propagate into the hollow ring “R” shown in FIG. 11 without substantial redirection, so that substantially most or all (e.g., 70%, more preferably 85%, and more preferably still upward of 95%) of the light incident on the lens 100 is focused into the hollow outer ring. In one example, the width “W” of the ring refers to the width of the ring in the focal plane of the lens 100, which typically can be anywhere from a fraction of a centimeter to several centimeters behind the lens to coincide with the expected location of the patient's peripheral vision receptors when the frame on which the lens (typically, left and right lenses) is supported.

To accomplish this and referring back to FIG. 10, as shown the spacing “S” between adjacent concentric Fresnel ridges 102 may become progressively less from the perimeter 106 of the lens 100 to the center 108 of the lens. Also, as best shown in FIG. 10, the slopes or tangents (relative to the axis of light entering the lens) of the curvilinear non-vertical sides 110 of the ridges 102 may become progressively steeper, ridge to ridge, from the perimeter 106 of the lens 100 to the center 108 of the lens, with the ridge 102a nearest the center 108 having the steepest non-vertical side 110 slope “S1” and the ridge 102b nearest the perimeter 106 having the shallowest non-vertical side 110 slope. As shown by registration lines 112, the curvatures of the non-vertical sides 110 of the ridges 102 may vary according to the curvature of the surface 22 of the cuspate lens shown in FIGS. 2 and 3 at the same radial location on the cuspate lens as the Fresnel ridge is on the Fresnel lens 100. The curvature of the slopes of the non-vertical sides 110 of the ridges may be established using the equations in the '484 patent.

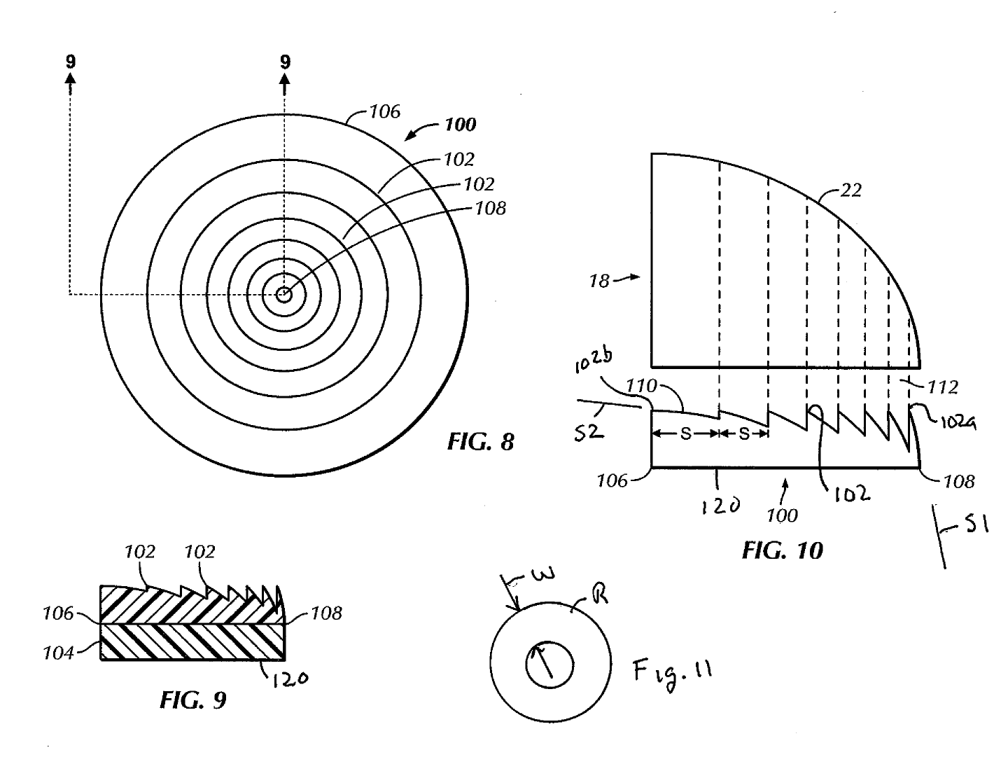

Note further in looking at FIGS. 9 and 10 that the peaks of the ridges 102 are substantially (e.g., within a millimeter or two) co-planar with each other, and that the plane in which the peaks of the ridges 102 lie is parallel to the plane defined by the smooth, flat output side 120 of the lens 100.

While the particular EYEWEAR TO ALLEVIATE AFFECTS OF MACULAR DEGENERATION is herein shown and described in detail, it is to be understood that the subject matter which is encompassed by the present invention is limited only by the claims.

1-16. (canceled)

17. Lens comprising:
substrate defining a center, a perimeter, and no material beyond the perimeter;
concentric rings of Fresnel ridges formed on the substrate and no other light redirecting structure being formed on the substrate, the substrate not being a portion of a larger lens structure, a spacing “S” between adjacent concentric Fresnel ridges becoming progressively less from the perimeter of the lens toward the center of the lens, slopes relative to an axis of light entering the lens of curvilinear non-vertical sides of the ridges becoming progressively steeper, ridge to ridge, from the perimeter of the lens to the center of the lens, such that light entering the lens is diverted into a hollow ring-shaped pattern of light exiting the lens, wherein a ridge nearest the center has a steepest non-vertical side slope relative to remaining ridges and a ridge nearest the perimeter has a shallowest non-vertical side slope relative to remaining ridges, and the respective slopes of all respective ridges relative to the axis of light become progressively steeper ridge to ridge in a direction from the ridge nearest the center to the ridge nearest the perimeter.

18. (canceled)

19. The lens of claim 17, wherein the substrate is flexible and is holdable onto an outer surface of a conventional eyeglass lens by adhesive or by simple friction/static charge.

20. Lens assembly, comprising:
concentric rings of Fresnel ridges formed on a thin flexible substrate configured for being held onto an outer surface of an eyeglass lens by adhesive or by simple friction/static charge, the Fresnel ridges having a configuration such that all light impinging at and near the center of the lens is redirected radially outwardly into a hollow ring, light impinging on outer portions of the lens being allowed to propagate into the hollow ring without substantial redirection, wherein no other light redirecting structure apart from the Fresnel ridges which redirect all light impinging at and near the center of the lens radially outwardly into a hollow ring is formed on the substrate, the substrate not being a portion of a larger lens structure, all light impinging at and near the center of the lens is redirected radially outwardly into a hollow ring; and
human-wearable eyeglass frame supporting the thin flexible substrate.

21. The lens of claim 20, wherein the configuration of the Fresnel ridges focuses substantially most or all of the light incident on the lens into the hollow outer ring.

22. The lens of claim 20, wherein the configuration of the Fresnel ridges such that the width of the hollow ring substantially matches a remaining width of peripheral vision of a patient suffering from macular degeneration.

23. The lens of claim 20, wherein a spacing “S” between adjacent Fresnel ridges becomes progressively less from a perimeter of the lens toward the center of the lens.

24. The lens of claim 20, wherein slopes relative to an axis of light entering the lens of non-vertical sides of the ridges become progressively steeper, ridge to ridge, from a perimeter of the lens to the center of the lens.

25. The lens of claim 23, wherein slopes relative to an axis of light entering the lens of non-vertical sides of the ridges become progressively steeper, ridge to ridge, from the perimeter of the lens to the center of the lens.

1-16. (canceled)

17. Lens comprising:
substrate defining a center, a perimeter, and no material beyond the perimeter;
concentric rings of Fresnel ridges formed on the substrate and no other light redirecting structure being formed on the substrate, the substrate not being a portion of a larger lens structure, a spacing “S” between adjacent concentric Fresnel ridges becoming progressively less from the perimeter of the lens toward the center of the lens, slopes relative to an axis of light entering the lens of curvilinear non-vertical sides of the ridges becoming progressively steeper, ridge to ridge, from the perimeter of the lens to the center of the lens, such that light entering the lens is diverted into a hollow ring-shaped pattern of light exiting the lens, wherein a ridge nearest the center has a steepest non-vertical side slope relative to remaining ridges and a ridge nearest the perimeter has a shallowest non-vertical side slope relative to remaining ridges, and the respective slopes of all respective ridges relative to the axis of light become progressively steeper ridge to ridge in a direction from the ridge nearest the center to the ridge nearest the perimeter.

18. (canceled)

19. The lens of claim 17, wherein the substrate is flexible and is holdable onto an outer surface of a conventional eyeglass lens by adhesive or by simple friction/static charge.

20. Lens assembly, comprising:
concentric rings of Fresnel ridges formed on a thin flexible substrate configured for being held onto an outer surface of an eyeglass lens by adhesive or by simple friction/static charge, the Fresnel ridges having a configuration such that all light impinging at and near the center of the lens is redirected radially outwardly into a hollow ring, light impinging on outer portions of the lens being allowed to propagate into the hollow ring without substantial redirection, wherein no other light redirecting structure apart from the Fresnel ridges which redirect all light impinging at and near the center of the lens radially outwardly into a hollow ring is formed on the substrate, the substrate not being a portion of a larger lens structure, all light impinging at and near the center of the lens is redirected radially outwardly into a hollow ring; and
human-wearable eyeglass frame supporting the thin flexible substrate.

21. The lens of claim 20, wherein the configuration of the Fresnel ridges focuses substantially most or all of the light incident on the lens into the hollow outer ring.

22. The lens of claim 20, wherein the configuration of the Fresnel ridges such that the width of the hollow ring substantially matches a remaining width of peripheral vision of a patient suffering from macular degeneration.

23. The lens of claim 20, wherein a spacing “S” between adjacent Fresnel ridges becomes progressively less from a perimeter of the lens toward the center of the lens.

24. The lens of claim 20, wherein slopes relative to an axis of light entering the lens of non-vertical sides of the ridges become progressively steeper, ridge to ridge, from a perimeter of the lens to the center of the lens.

25. The lens of claim 23, wherein slopes relative to an axis of light entering the lens of non-vertical sides of the ridges become progressively steeper, ridge to ridge, from the perimeter of the lens to the center of the lens.
